(19) United States
(12) Patent Application Publication
(10) Pub. No.: US 2008/0138797 A1
(43) Pub. Date: Jun. 12, 2008

(54) Detection Systems and Methods

(76) Inventors: William D. Hunt, Decatur, GA (US); John Cairney, Decatur, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 10/579,009

(22) PCT Filed: Nov. 15, 2004
(86) PCT No.: PCT/US04/38021

§ 371 Date: May 16, 2007

Related U.S. Application Data
(60) Provisional application No. 60/519,800, filed on Nov. 13, 2003.

(51) Int. Cl. C12Q 1/68; C12M 1/34; B01J 19/00; G01N 21/01; C12N 5/06; C12M 1/36; G01N 33/00; C40B 40/00

(52) U.S. Cl. 435/6; 435/287.1; 435/286.1; 435/288.7; 435/287.2; 422/68.1; 422/82.05; 436/131; 436/73; 436/94; 435/395; 506/13

(57) ABSTRACT

Detection systems and methods of their use are provided. An exemplary system comprises a chamber for holding culture media, the chamber having a cellular attachment surface, and a detector disposed in the chamber comprising a surface modified with a binding agent for binding a target substance wherein the detection system is configured to detect interaction of the target substance with the binding agent. The detection can occur in either liquid or vapor phase and the subsequent action of the system is to respond in a programmed and appropriate manner to the binding event by activation of a chemical or physical responder. The system may also respond by communicating information to a control system via an alarm.

### CROSS REFERENCE TO RELATED APPLICATION

This application claims benefit of and priority to U.S. Provisional Patent Application No. 60/519,800 filed on Nov. 13, 2003, and where permissible is incorporated by reference in its entirety.

### BACKGROUND

1. Technical Field

The present disclosure is generally related to systems and methods for detecting target substances, in particular systems and methods that monitor real-time culture conditions.

2. Related Art

The biotechnology industry produces many therapeutic products including protein or peptide products produced from cells grown in laboratories. Using cells to produce therapeutic products can be problematic because the cells must be maintained in sterile conditions and must be constantly given appropriate levels of nutrients. Cell culture conditions generally must approximate physiological conditions for the cells to grow. Thus, the pH of cell culture media must be appropriately buffered and the temperature of the cell culture must be maintained. Under appropriate conditions, the cells growing in culture can secrete therapeutic proteins or other therapeutic molecules into the cell culture media. The therapeutic proteins can be collected from the cell media and concentrated or purified for use in a commercial product. The same principles hold for production in cell culture of molecules of commercial interest.

In addition to production of therapeutic molecules, cell cultures are being used to engineer complex cellular structures ex vivo. Such cell structures include tissues, valves, cartilage, blood vessels, organs or parts of organs. Recent advances in stem cells have enabled significant advances in producing these tissues from cell cultures. To form specific tissues or to differentiate into specific cell types, stem cells often require the interaction of other cells or substances secreted by other cells. Many growth factors and molecules that induce differentiation have been identified. These growth factors and induction agents can be applied to stem cells grown in culture to form a desired structure or tissue. The amount of a specific agent and the time the agent is applied to the cell culture are factors that can have a significant effect on stem cell differentiation.

In commercial bioreactors where cells are cultivated to produce biochemicals, optimal growth of a culture is required in order to maximize the production of desirable molecules. Impaired growth due to suboptimal culture conditions, which might arise where a culture lacked nutrients or oxygen or substrates for synthesis, will induce metabolic adjustments required for the cells to accommodate the changed growth conditions. The reallocation of substrate molecules and the redirection of energy into different pathways will have the effect of reducing flux through the pathway of interest to the manufacturer. Such metabolic alterations can often be detected by the appearance of proteins or other molecules that are associated with ‘stress responses’ or by appearance of molecules associated with alternative metabolic pathways, for example, those associated with anaerobic growth or the utilization of reserve energy sources. The detection of such ‘marker’ molecules can alert a manufacturer to the physiological status of the cell culture and permit remedial action to be taken, which with restore the culture to optimal growth and optimal production of the molecules of interest.

Accordingly, there is a need for systems and methods that can monitor the presence or absence of specific substances, for example the detection a target substances in cell cultures.

### SUMMARY

Aspects of the present disclosure generally provide detection systems and methods of their. An exemplary system comprises a chamber for holding culture media, the chamber optionally having a cellular attachment surface, and a detector disposed in the chamber comprising a surface modified with a binding agent for binding a target substance wherein the detection system is configured to detect interaction of the target substance with the binding agent. The detection can occur in either liquid or vapor phase, and the subsequent action of the system can be to respond in a programmed and appropriate manner to the binding event by activation of a chemical or physical responder. The system may also respond by communicating information to a control system via an alarm.

In some aspects, the detector comprises a piezoelectric substrate surface-modified with a binding agent for binding the target substance and a pair of electrodes coupling the piezoelectric substrate to an operating system. In other aspects, the detector is selected from an optical detection device, MEMS detection device (e.g. cantilevers and micromachined resonating structures, nanoparticle detection device (e.g. quantum dots or quantum piezoelectric dots), spectroscopic techniques or an acoustic wave detection device.

Other aspects provide systems and methods for detecting a target substance in culture media, on the surface of cells, in ambient atmosphere, and in pulping systems. Still other aspects provide systems and methods for detecting target substances in real-time, for example gene expression profiles.

### BRIEF DESCRIPTION OF THE FIGURES

FIG. 1 shows an exemplary embodiment of a cell culture system according to the present disclosure.

FIG. 2 shows another exemplary embodiment of a cell culture system according to the present disclosure.

FIG. 3 shows an exemplary array for detecting biomolecules according to the present disclosure.

FIG. 4 shows a flow diagram of an exemplary method of detecting a target substance according one embodiment of the present disclosure.

Fig. is a schematic of an exemplary method for fixing antibodies to the QCM surface.

FIG. 6 shows a line graph showing the detection of calmodulin with an exemplary system according to one embodiment of the disclosure

FIG. 7 shows a line graph of frequency change vs. injected number of B. subtilis spores detected with an exemplary system of the present disclosure.

### DETAILED DESCRIPTION

Embodiments of the present disclosure provide systems and methods for detecting target substances. Exemplary methods and systems include systems for detecting the presence or absence of a target substance, more particularly for detecting the presence or absence of a target substance in a cell culture, and optionally responding to the detection of the target substance. One embodiment, among others, provides a biosensor configured to detect the presence or absence of a target substance. Though most of the discussion to follow implies detection in a liquid phase medium, the various embodiments described herein are not limited to detection in the liquid phase. Some embodiments detect target substances in the vapor phase. Exemplary target substances include pathogens such as molds or other infectious or colonizing life forms, as well as allergens, and contaminants.

In general, the disclosed systems detect a target substance and optionally respond to the presence of the target system. An exemplary response includes, but is not limited to the automated release of an agent. for example a growth factor or a disinfecting agent. In some embodiments, the response includes providing a disinfecting amount of electromagnetic radiation such as ultraviolet light to incapacitate, inactivate or kill a pathogen or target substance detected in the system or in the vicinity of the system. The system can also respond in a programmed and appropriate manner to the binding event by activation of a chemical or physical responder. The system may also respond by communicating information to a control system via an alarm.

A particular embodiment provides a biosensor that can intermittently or continuously monitor the contents of a cell culture, for example substances in the cell culture media or substances displayed on cell surfaces. Continuous monitoring is also referred to as monitoring or detection in “real-time”. Intermittent monitoring generally refers to periodic monitoring. Such periodic monitoring can occur at regular intervals or irregularly. Periodic monitoring or detection typically occurs about 1 to about 10 times per hour, per minute, or per second. The frequency with which sampling and detection occurs will vary with the demands of a particular application. For example, application of the approach in the field of tissue engineering would require a much smaller time scale for monitoring and response than would, say, the fermentation of wine. For tissue engineering one is attempting to mimic the rapid, massively parallel development of an organ that is found in vivo.

In some embodiments, the detection of a target substance is accomplished using a detector which could be any one of a wide variety of modalities—optical, surface plasmon resonance, acoustic cantilever or any one of an emerging group of MEMS and nanotechnology approaches such as cantilevers, micromachined resonating structures, nanoparticle detection device such as quantum dots or quantum piezoelectric dots, spectroscopic techniques or an acoustic wave detection device. An exemplary detector comprises a piezoelectric substrate surfaced-modified with a binding agent specific for the target substance. Exemplary piezoelectric substrate materials include, but are not limited to quartz (SiO₂), LiTaO₃, LiNbO₃, GaAs, SiC, LGS, ZnO, AIN, PZT, PVdF, or a combination thereof.

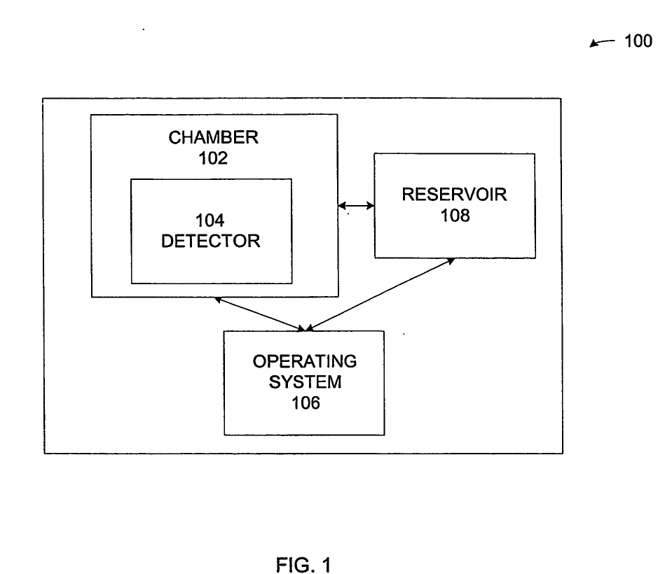

FIG. 1 shows a high level diagram of a representative system 100 according to one embodiment of the present disclosure. System 100 generally includes a chamber 102 for holding culture media, typically liquid culture media, and cells. At least one piezoelectric substrate or piezoelectric detector 104 is disposed in the chamber for monitoring or detecting the presence or absence of a target substance. A representative piezoelectric detector 104 includes, but is not limited to an acoustic wave detector or sensor. An exemplary acoustic wave detector comprises a piezoelectric substrate, an input transducer and an output transducer. Such detectors can also be a so-called one-port device wherein there is only a solitary pair of inputs to the sensor and the reading of the detector is done by inclusion of the detector into a circuit where the changes in the electrical impedance of the detector modifies circuit characteristics which can be easily measured electronically. In one embodiment, system 100 is configured to detect at least about 1 to about 1000 attograms of a target substance, typically at least about 50 to about 500 attograms of a target substance.

Acoustic wave detectors typically operate by detecting changes in characteristics of an acoustic wave as the acoustic wave travels through or on the surface of a piezoelectric substrate. Applying an appropriate electrical field on a piezoelectric substrate creates a mechanical stress on the substrate. The acoustic wave sensors or detectors generally apply an oscillating electric field to a piezoelectric substrate to create a mechanical wave which propagates on the surface or through the substrate and is converted back to an electric field for measurement or detection. Obstacles in the path of the acoustic wave will alter the velocity and/or amplitude of the acoustic wave. Changes in wave velocity can be monitored by measuring the frequency or phase characteristics of the piezoelectric substrate component of the acoustic wave detector.

A variety of acoustic wave detectors can be used with the disclosed systems. Generally, acoustic wave detectors are described by the mode of wave propagation through or on the piezoelectric substrate. A wave propagating through the substrate is referred to as a bulk wave. Representative bulk wave devices include, but are not limited to the thickness shear mode (TSM) resonator and the horizontal acoustic plate mode (SH-APM) sensor. The TSM resonator is the configuration utilized for the Quartz Crystal Microbalance (QCM).

If the wave travels on the surface of the piezoelectric substrate, the wave is referred to as a surface wave. Exemplary acoustic wave devices using surface waves include, but are not limited to surface acoustic wave (SAW) sensor and the shear-horizontal surface acoustic wave (SH-SAW) sensor also referred to as the surface transverse wave (STW) sensor.

Because the TSM, SH-APM, and SH-SAW generate waves that propagate primarily in the shear horizontal motion, these acoustic wave sensors are well suited for use with the systems and methods of the present disclosure. Acoustic wave detectors or sensors that use waves that propagate at a velocity lower than the sound velocity in liquid are also particularly useful in the disclosed systems and methods.

FIG. 1 further shows piezoelectric substrate 104 in communication with operating system 106 which is in turn in communication with optional reservoir 108. Operating system 106 includes, but is not limited to, electronic equipment capable of measuring characteristics of a target substance, for example a polypeptide, as is interacts with piezoelectric substrate 104, a computer system capable of controlling the measurement of the characteristics and storing the corresponding data, control equipment capable of controlling the cell culture conditions and piezoelectric substrate 104, and components that are included in piezoelectric substrate 104 that are used to detect, measure or quantify the presence or absence of a target substance in chamber 102. Bioreactor system 100 can also be in communication with a distributed computing network such as a LAN, WAN, the World Wide Web, Internet, or intranet.

FIG. 1 also shows reservoir 108 communicatively connected to operating system 106. Reservoir 108 generally contains cell culture reagents such as liquid media, nutrients, fetal calf serum, antibiotics, pH buffer, acid, base, growth factors, differentiation inducing agents, apoptosis inducing agents, protein synthesis inhibitors, microtubule stabilizers, and translation and transcription inhibitors. The contents of reservoir 108 can be released into chamber 102 as needed or as determined by operating system 106. Alternatively, reservoir 108 can be a sample processing chamber. The sample processing chamber can remove substance that may interfere with detection of the target substance, concentrate a sample, modulate the temperature or pH of a sample, or otherwise optimize a sample for detecting the target substance.

The biosensor device can also be placed within a bioreactor, to monitor the medium directly for example by submersing the biosensor in culture media. Alternatively, the biosensor device be outside the chamber and samples from the bioreactor or a designated reservoir tank can be analyzed remotely.

To extend the use of the biosensor device to permit monitoring of media whose composition or condition differs greatly from a norm, for example media exhibiting extremes of pH or temperature or media highly enriched in a particular substance, a ‘conditioning chamber’ may be placed upstream of the biosensor device. The purpose of the conditioning chamber would be to modify the original sample in such a way as to optimize detection of the desired molecule by the biosensor device. This may be achieved in a number of ways, for example by cooling, altering media pH, or extracting a substance. The ‘conditioning’ process would be constructed such that the function and accuracy of the biosensor was optimized.

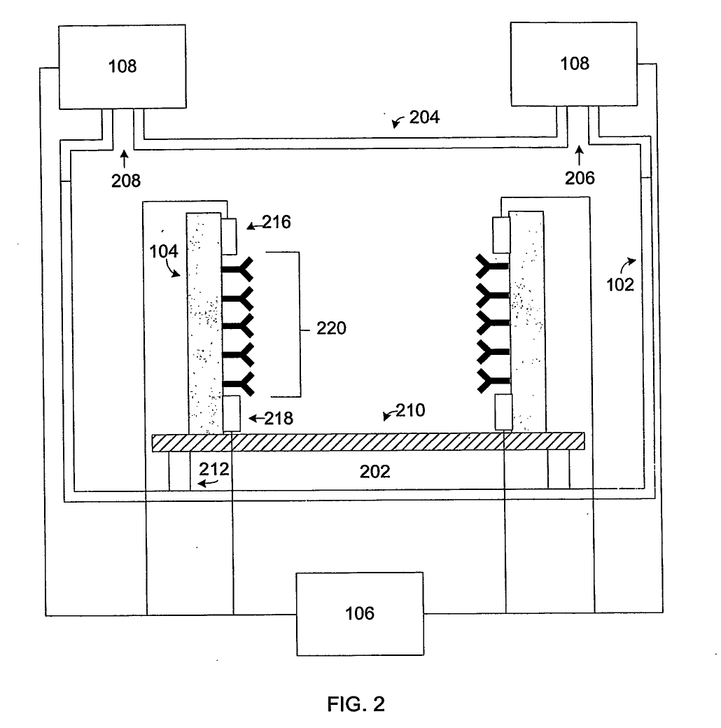

FIG. 2 shows another exemplary embodiment of a cell culture system or bioreactor according to the present disclosure. System 200 includes chamber 102, which optionally includes a removable cover 204. Chamber 102 is generally made from a polymer, plastic, thermoplastic, acrylic, acrylate, but it will be appreciated that any liquid impermeable substance can also be used. Cover 204 can be made from the same material as chamber 102, or alternatively, cover 204 can be made of a material that is gas permeable. The gas permeable material can be polymer or plastic optionally containing pores or apertures. The pores typically have a diameter that will not permit bacteria or spores to pass through into the chamber 102. Such pores can have a diameter of less than about 0.2 μm in diameter.

Cover 204 optionally includes one or more ports 206 and 208 which can be in fluid communication with one or more reservoirs 108. As noted above, reservoir 108 can contain material to be introduced into chamber 102. Ports 206 and 208 can be controlled by operating system 106 so that a desired material can be introduced from reservoir 108 into chamber 102 at a specific time or times in specific amounts. The ports can be positioned so that material introduced into chamber 102 does not flow directly onto optional cell attachment surface 210.

Cellular attachment surface 210 can be a solid or porous membrane, a three dimensional scaffold, glass, metal, plastic, polymer, thermoplastic, nylon, polysiloxane, acrylic, acrylate, or a combination thereof. The scaffold can be composed of cartilage. collagen, hydrogel, proteoglycans, plastic, polymers, or a combination thereof. Attachment surface 210 can be coated with a substance to facilitate cellular attachment, for example polylysine or positively charged substances. Generally, cellular attachment surface 210 is composed of a non-conductive substance. In one embodiment, cellular attachment surface 210 forms the bottom of chamber 102. In another embodiment, cellular attachment surface is elevated above the bottom of chamber 102, for example by one or more posts or columns 212.

Generally, cellular attachment surface 210 is elevated when composed of a porous membrane material, for example a porous nylon or nitrocellulose membrane. When elevated above the bottom of chamber 102, a subchamber 202 can be formed between cellular attachment membrane 210 the bottom of chamber 102. Subchamber 202 can contain fluid comprising a diffusible substance that traverses cellular attachment membrane 210 and enters chamber 108. The diffusible substance can be detected by at least one detector 104 disposed in chamber 108.

Detector 104 of system 200 comprises piezoelectric substrate 214. Input and output transducers 216 and 218 connect piezoelectric substrate 214 to operating system 106. Input transducer 216 introduces an electric field into piezoelectric substrate 214 to produce an acoustic wave. The acoustic wave is converted back to an electric field by output transducer 218. At least a portion of a surface of piezoelectric substrate 214 is modified with one or more binding agents 220 for interacting with a target substance. Exemplary binding agents include polypeptides, nucleic acids, antibodies, carbohydrates, lipids, receptors, or ligands of receptors, fragments thereof, and combinations thereof.

The generation of antibodies, including monoclonal, chimeric, and humanized antibodies, is well known in the art. The polypeptides, their fragments or other derivatives, or analogs thereof, or cells expressing them can be used as an immunogen to produce antibodies thereto.

These antibodies can be, for example, polyclonal or monoclonal antibodies. The present disclosure also includes chimeric, single chain, and humanized antibodies, as well as Fab fragments, or the product of an Fab expression library or antibodies to which additional molecules are attached. These modified antibodies can include but not be restricted to chimeric antibody molecules possessing an additional moiety or antibody molecules which function in close association with other molecules. Various procedures known in the art may be used for the production of such antibodies and fragments.

Techniques for attaching biomolecules to surfaces are known in the art. For example, a glass, silica, or quartz surface can be amino-silylated using a 2% solution of 3-aminopropyltriethoxysilane in acetone. The term “biomolecule” refers to a substance produced by a living organism or modulates a biological function of an organism, and includes but is not limited to polypeptides, polynucleotides, carbohydrates, lipids, vitamins, co-factors, chemical modifications and derivatives thereof. Biomolecules having an amine group can be linked to the silylated surface using a crosslinking agent such as sulfo-LC-SPDP. The biomolecule can be attached directly to the surface or indirectly through a cleavable linker molecule. The linker molecule can contain a photocleavable bond or a cleavage site recognized by an enzyme. In one embodiment, piezoelectric substrate 214 is modified with at least two different binding agents that specifically interact with two different target substances. Alternatively, at least two detectors 104 specific for different target substances respectively can be disposed in chamber 102.

When a target substance interacts with binding agent 220, characteristics of the acoustic wave traveling on or through piezoelectric substrate 214 change. This change can include a change in wave velocity or amplitude which can be detected and processed by operating system 106. In response to detecting the presence or absence of a target substance, operating system 106 can modify cell culture conditions by opening or closing port 206 or 208 to introduce or stop the introduction of material from reservoir 108.

The interaction of target substance with the binding can also induce one or more changes in either the target substance or the binding agent. Exemplary changes include, but are not limited to, changes in conformation, activation, cleavage of the target substance or binding agent, covalent modification of the target substance or binding agent, degradation of the target substance or the binding agent, formation of a reaction product from the interaction of the target substance with the binding agent, or combinations thereof. For example the binding agent can be an enzyme and the target substance can be a substrate of the binding agent. Alternatively, the target substance can be an enzyme and the binding agent can be a substrate of the enzyme. Interaction between the enzyme and substrate could produce additional molecular species or products. The products of the enzymatic interactions can be growth factors, cytokines, differentiation inducing factors, or combinations thereof. Thus, the presence of a target substance can trigger the release of an inducing agent produced by the interaction of the target substance with the binding agent. This interaction can also be detected by the detection system, for example as a change in frequency of the piezoelectric substrate.

In another embodiment, the interaction of a target substance with the binding agent can modify the target substance, binding agent, or both, for example by inducing structural changes in the target substance, or by tagging the target substance with a detectable label or with a second binding agent. The modified target substance can then interact with a second binding agent. The modified binding agent can be changed so that it can no longer interact with the unmodified target substance or can interact with a second target substance. In this embodiment, an increase in specificity can be achieved because the first target substance must be present before the modified target can be detected. As noted above, the modified target substance can be a growth factor or detectable reaction product. In still another embodiment the reaction product is detectable using fluorometric detection, colorometric detection, or mass spectroscopy detection methods.

In another embodiment, the interaction of target substance with the disclosed detector system induces changes in either the target substance or the binding agent so that the modified molecules cannot interact with one or more components of the detector system. For example, the interaction of target substance with the detector system can degrade, remove the target substance from the detector system, or make the modified target substance unavailable for example by sequestering or covalently binding the modified target substance. The removal of the target molecule may be part of the harvesting of target molecules. Moreover, the removal may be a means of maintaining cell culture conditions within prescribed parameters, for example by removing a cytotoxin or other molecule which may shift the culture conditions or physiological status of the cells from their optimal range.

It will be appreciated that the target substance can be any detectable substance, and typically is a biomolecule. Examples include but are not limited to cell surface receptors such as membrane bound kinases or ion channels, secreted substances such as arabinogalactan proteins or iron scavenging proteins, regulatory molecules such as calmodulin and fragments of these molecules, cell derived carbohydrates, lipid moieties, ‘stress’ or ‘defense’ molecules, products of secondary metabolism, molecules associated with programmed cell death, molecules that are produced by cells as the result or genetic engineering or growth regulation. In one embodiment, the interaction of the target substance with the binding agent is monitored in real-time. In another embodiment, the interaction is monitored periodically, for example every hour, typically, every 1-5 minutes, more typically about every minute.

Exemplary cells that can be cultured with the disclosed systems and methods include but are not limited to eukaryotic, archaebacterial and prokaryotic cells. The eukaryotic cells or archaebacterial cells or prokaryotic cells can be transfected with a polynucleotide, for example to express a polypeptide. Eukaryotic cells include fungi, animal, and plant cells, and prokaryotic cells include bacteria and archaebacteria. Exemplary animal cells include, but are not limited to, primary culture cells, stem cells, embryonic cells, embryonic stem cells, adult stem cells, bone marrow stem cells, pluripotent cells, somatic cells, tissues, organs, transfected cells, immortalized cells, non-transformed cells, transformed cells, or combinations thereof. Exemplary fungi cells include, but are not limited to yeast. Archaebacteria, often isolated from environmentally harsh conditions, are noted for their tolerance of extremes of pH, temperature, pressure, heavy metals, and many other conditions. The physiology of archaebacteria is being exploited to permit production of chemicals under conditions optimal for certain manufacturing or treatment processes but inhibitory or fatal to most eukaryotic and prokaryotic cells. For example tolerance of high temperatures has led to the use of certain archaebacteria or enzymes derived from archaebacteria in manufacturing and treatment processes. The biosensor device described here can be deployed to monitor bioprocesses in archaebacterial bioreactors or treatment systems.

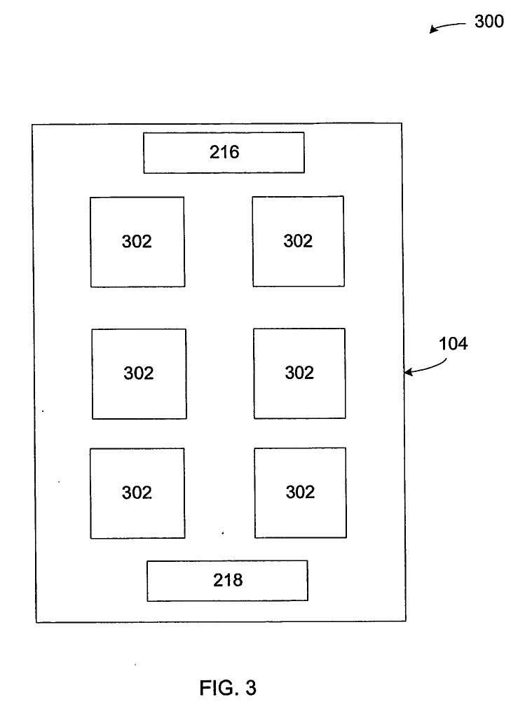

FIG. 3 shows an exemplary piezoelectric array 300. Array 300 includes a piezoelectric substrate 104 having input transducer 216 and output transducer 218. At least two regions 302 of a surface of the piezoelectric substrate 104 are modified by attaching, connecting, adsorbing, absorbing, coating, or otherwise applying a binding agent. Generally, each region contains a binding agent that specifically binds a different target substance. One or more types of binding agents can be used in a single region 302 or a different type of binding agent can be used in each region 302. For example, one region can include antibodies, whereas another region can include polynucleotides. Alternatively, one region can contain more than one type of binding agent. The array can include an operating system 106 for controlling the transducers and storing data. Specific binding patterns in the array can be correlated with specific stages of cell culture growth or cell differentiation or production or removal of specific molecules by the cell culture. The biosensor may detect the target molecules directly or indirectly through the involvement of another molecule which may or may not be in close association with the biosensor.

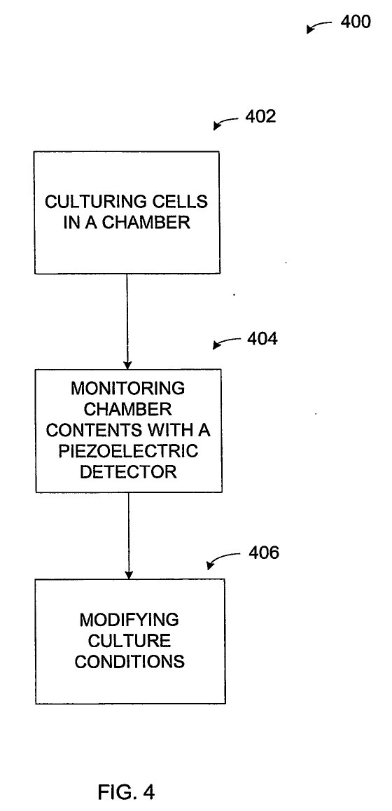

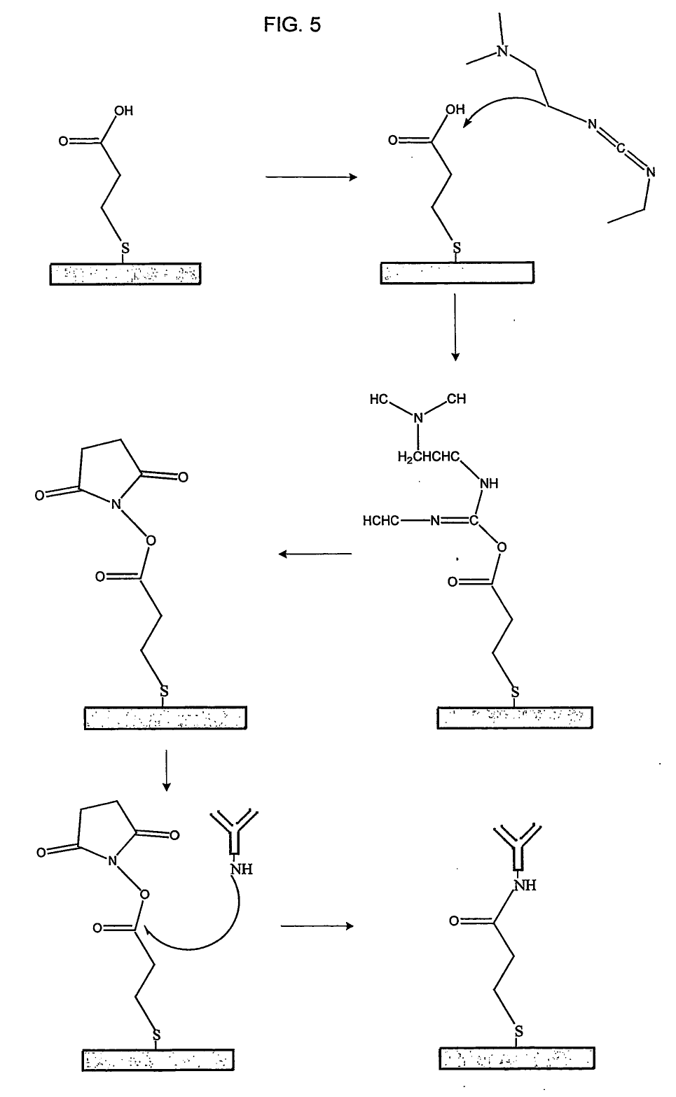

FIG. 4 shows a flow diagram of an exemplary method according to the present disclosure. In process 400, cells are cultured in a chamber. A piezoelectric substrate surface-modified with at least one binding agent can be used to detect the presence or absence of a target substance in the culture chamber. Generally, the target substance to be detected is a substance produced by the cells being cultured. For example, a specific protein or polypeptide secreted or released by the cells in culture can be detected. The presence of the polypeptide can then be correlated with the occurrence of a particular event, a stage of growth, a stage of maturity, a stage of differentiation, or the production of a desired product from a recombinant cell. Once the target substance is detected, the cell culture can be selected for further processing, or the conditions of the cell culture can be optimized for increased growth or increased product production. For example a cell culture may be monitored, and when a chosen stage of development or cell density is attained a known marker molecule is produced by the cells, this marker molecule is detected by the biosensor, the biosensor then can alert the system operator, or alternatively, the biosensor can be programmed to open a valve to allow addition of an inducer or similar effector molecule to the culture which then causes the cells to respond in a desired way (such as by commencing production of the molecules of interest). Similarly the biosensor can by used to monitor growth conditions and maintain them within a specified range, again by detecting marker molecules which are either produced at a given level within the growth range or are produced when the culture moves outside of the specified growth conditions. When the biosensor detects a specified change it can be programmed to activate set responses in the system or can be programmed to alert the system operating staff.

In one embodiment plant cells, for example loblolly pine embryos, are cultured and the disclosed systems are used to monitor for the presence of a biological marker correlated with a desired cellular or plant characteristic. An exemplary biomarker includes, but is not limited to Somatic Embryogenesis Receptor Kinase (SERK), Aribinogalactan proteins (AGPs), PtFIE, PtABI3, PtLEC1, PtPKL, PtPNHD, EP3, PKL, LEC, ABI3, CLAVATA1-3 and orthologs or homologues thereof. A biomarker can be selected to differentiate between robust cell cultures and cell cultures that will develop inferior plants. Cultures in which the biomarker is detected will be selected, and cultures in which the biomarker is not detected are discarded.

Similarly, differentiation of a culture of undetermined cells, for example stem cells or pluripotent cells can be controlled using the disclosed systems and methods. Undifferentiated cells can be cultured in a chamber having a piezoelectric substrate surface modified to detect the expression of polypeptides indicative of a specific cell type, tissue type, or stage of development. The chamber optionally includes a scaffold. As the cells are cultured, the contents of the cell culture media or the expression of a specific biomarker can be monitored in real-time. The data can be recorded and processed by an operating system. Based on the substances detected in the culture media and the cell type desired, the operating system can trigger the release of one or more agents known to induce cellular differentiation into a specific cell type. Exemplary factors that are known to induce differentiation include, but are not limited growth factors, mitogens, platelet-derived growth factor (PDGF-AA, -AB, and -BB0, bone morphogenic proteins 1-14, noggin, noggin-like proteins, chordin, VEGF, stem cell factor, extracellular signal-regulated kinase (ERK), EFG, FGF, FGF-2, insulin, notch, LIF, CNTF, SHH, cytokines, IL-1, IL-2, IL-3, IL-4, IL-5, IL-6, IL-7, IL-8, IL-9, IL-10, IL-11, IL-12, IL-13, neutrophines, Rho protein, G-CSF, GM-CSF, TGF-α, TGF-β, TNF-α, TNF-β, IGF-I, IGF-II, INF-α, INF-β, INF-γ, and combinations thereof. Additionally, cell density can be modulated to promote the formation of a specific cell type or tissue.

FIG. 5 shows an exemplary method for modifying the surface of a detection device, for example a piezoelectric substrate surface. In this method, the modified surface comprises a surface having a compound attached to the surface through a thiol linkage. A representative surface includes, but is not limited to a metal surface such as a gold surface. The gold surface is typically layered or deposited on a piezoelectric substrate. FIG. 5 shows a surface modified with 3,3′-dithiopropionic acid. It will be appreciate that any thiolcarboxylic acid can be used, for example thiocarboxylic acids having branched or unbranched alkyl chains from about 3 to about 12 carbons. Carbodiimide coupling can then be performed using, for example, 1-Ethyl-3-(3-Dimethylamino-propyl) carbodiimide (EDC), optionally in the presence of N-hydroxysuccinimide (NHS). In the reaction EDC converts the carboxylic acid into a reactive intermediate which is susceptible to attack by amines. A binding agent having an amine group can then be attached to the piezoelectric substrate by linking to the reactive intermediate. Representative binding agents include, but are not limited to, polypeptides, nucleic acids, peptide nucleic acids, enzymes, enzymatic nucleic acids, nucleic acids having modified backbones, carbohydrates, lipids, vitamins, and small organic molecules.

In FIG. 5, the binding agent is an antibody. It will be appreciated that the antibody can be any type of antibody including, but not limited to a mouse, sheep, goat, horse, guinea pig, rabbit, mammalian, human, or primate. Antibodies generated against the polypeptides corresponding to a sequence of the present disclosure can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler, G. and Milstein, C., Nature 256: 495-497 (1975), the trioma technique, the human B-cell hybridoma technique (Kozbor et al., Immunology Today 4: 72 (1983) and the EBV-hybridoma technique to produce human monoclonal antibodies (Cole et al., pg. 77-96 In Monoclonal Antibodies and Cancer Therapy, Alan R. Liss, Inc. (1985).

Techniques described for the production of single chain antibodies (U.S. Pat. No. 4,946,778) can be adapted to produce single chain antibodies to immunogenic polypeptide products of this disclosure. Also, transgenic mice, or other organisms such as other mammals, may be used to express humanized antibodies to immunogenic polypeptide products of this disclosure.

### The above-described antibodies may be employed to detect a specific target substance of class of target substances sharing a common epitope.

One embodiment of the present disclosure provides an antibody that specifically binds to a specific epitope of a target substance. In another aspect, the antibody recognizes a protein complex, but does not significantly recognize the individual proteins forming the complex. The antibodies can be monoclonal, polyclonal, chimeric, humanized, single chain or fragments thereof including Fab fragments.

An alternative method for modifying the piezoelectric substrate surface employs dithiobis(N-succinimidyl propionate) (DTSP) also known as Lomant's reagent can be used to modify a piezoelectric substrate. DTSP adsorbs onto gold surfaces through the disulfide group, so that the terminal succinimidyl groups allow further covalent immobilization of amino-containing organic molecules or enzymes. In some embodiments, the piezoelectric surface is further modified with a layer of hydrogel over the immobilized antibodies to provide a near-aqueous environment necessary for maintenance of the tertiary structure of these biomolecules.

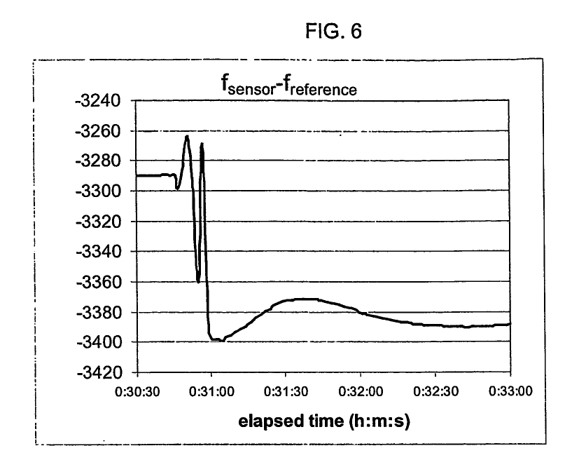

FIG. 6 shows an exemplary biosensor response to the addition of 100 μL Calmodulin at about 5 μg/ml concentration (500 ng in solution). The approximate detection limit in this particular example, with 0.1 Hz noise level, is 417 pg. Anti-Calmodulin antibodies (Anti-CaM Abs) were obtained from Abcam biochemicals, (Cambridge Mass. Cat#1288). Anti-CaM antibodies were tethered to the device using an alkane-thiol self assembled monolayer (SAM) protocol (summarized in FIG. 5) One milliliter of Tris-EDTA buffer (pH 7.6) was added to the detector chamber and the system was allowed to equilibrate. Purified Calmodulin peptide (Abcam biochemicals, Cat. No. ab5015) was added to a final quantity of 500 nanograms. The response of the biosensor device was recorded in real-time on a laptop computer to which the sensor device and associated electronics were connected. Other embodiments of the device allow detection limits in the attogram range. The exemplary system described above has also been used successfully in a complex growth medium at pH 5.

Further, in FIG. 6 the real-time nature of the detection approach is demonstrate. FIG. 6 shows the frequency shift as a function of time minus the frequency shift of the reference sensor. The data shown in FIG. 6 demonstrate that the disclosed systems can detect changes due to mass attachment to the acoustic sensor as well as conformational changes of the antibody film as well. Accordingly, embodiments of the disclosed systems include real time detection of binding events as well as determination of the binding affinity of the target substance the immobilized binding agent.

In yet another embodiment, two target substances can be distinguished based on the data obtained when the target substances interact with the detector. For example, one target substance can induce a conformational change in addition to a change in mass. The conformational change in combination with a mass change can generate a unique data signature. Other target substances will not induce a conformational change, and therefore will have a data signature that is different from target substances that do induce a conformational change. The conformational change can be in either the biomolecule or binding agent.

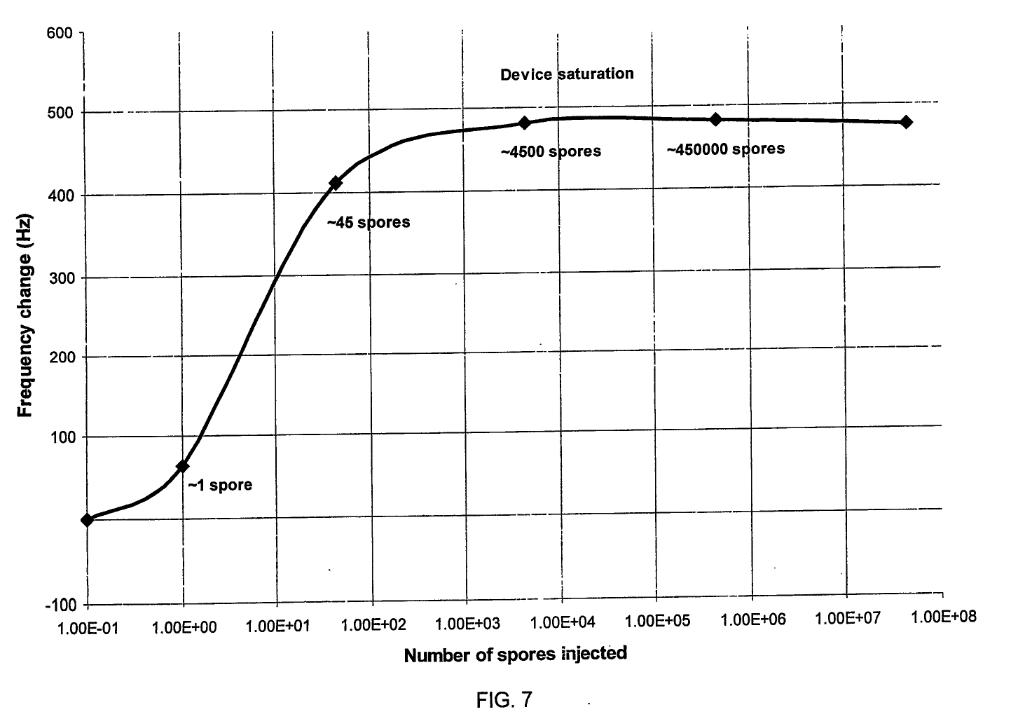

FIG. 7 shows the dose-response curve for an exemplary biosensor comprising an acoustic sensor (QCM) coated with an antibody specific for spores of Bacillus globigii The antibody was obtained from Dr. John Kearney of the University of Alabama Birmingham Medical School. Serial dilutions of spores were made in Tris-EDTA buffer (pH 7.6) and introduced in to the detection chamber. The response of the biosensor device was recorded in real-time on a laptop computer to which the sensor device and associated electronics were connected. In order to obtain the frequency values shown, the asymptotic frequency value for each presentation of spores was recorded. FIG. 7 represents an abstract of a large body of data generated by an exemplary approach according to the present disclosure and presents the results in a conventional dose-response curve form. The curve shows the net frequency shift versus spore concentration and hence demonstrates the dynamic range of the sensor. As can be seen from this curve the detection limit available is down to the level of a single spore. The spores were obtained from Dr. Alex Hoffmaster of the Centers for Disease Control in Atlanta, Ga.

In another embodiment, the disclosed systems and methods can be used to maintain levels of specific substances in cell culture media. For example, the disclosed systems can be configured to continuously monitor levels of a target substance in the culture. The target substance can be one or more growth factors or differentiation inducing agents. When levels of a target substance decrease, the system can respond by releasing additional target substance into the culture chamber. Maintaining a consistent level of nutrients or target substances can provide a greater degree of control in tissue engineering.

Yet another embodiment provides systems and methods for the detection of substances associated with or markers of scaling problems in pulping systems, for example Kraft pulping systems. An exemplary substance or scaling agent includes, but is not limited to hexenuronic acid (HexA), catechol or catechol containing structures (to correlate with burkeite), and aluminum sulfate (to correlate with barium sulfate scale). HexA is principally found in the bleach line of pulping systems.

Kraft pulping industries have been progressively focused on major process changes such as improved wood handling, new methods of modified cooking, the use of non-chlorine bleaching chemicals and closed system processes. One of the important steps to accomplish a close system is to eliminate the effluent from the bleaching plant discharge into receiving water. However, a closed system may affect the chemical consumption and pulp quality due to carry over of organic and inorganic components within the plant resulting in scale deposits.

One source of the scale is from the formation of oxalic acid, in particular calcium oxalate, during Kraft cooking and bleaching processes. One source of oxalic acid is from the native wood, which can contain about 0.1-0.4 kg oxalic acid/t and from the oxidation reaction of the residual of hexenuronic acid (hexA) in the pulp.

Accordingly, one embodiment provides an online or inline HexA, catechol, or aluminum sulfate detection system comprising a detector surface-modified with a binding agent, for example an antibody, specific for HexA, catechol, or aluminum sulfate. An exemplary detector can include a piezoelectric substrate. The detection system can be in fluid communication with the pulping system so that continuous or periodic samples of the pulping system can be delivered to the detection system. Generally, the detection system comprises one or more binding agents for binding one or more scaling agents. The interaction of a scaling agent with the binding agent will result in a change in frequency of the piezoelectric substrate. The change in frequency can be correlated with the presence of a specific scaling agent in the sample.

The detection system can be configured to detect one or more scaling agents or predetermined levels of one or more scaling agents. A scaling agent refers to any molecule or substances known or suspected of contributing either directly or indirectly, to the formation of scale deposits. Scale refers to a water insoluble material formed by one or more substances including, but not limited to sulfates, oxides, carbonates, salts, metals, organic compounds, minerals, and combinations thereof.

In still another embodiment, the disclosed detection system can be configured to detect a airborne or aqueous pathogens. Exemplary pathogens include bacteria, fungus, virus, protozoa, mycoplasma, parasites, spores, or combinations thereof. An exemplary detector system comprises a detector. The detector can include substrate, for example a piezoelectric substrate, surface-modified with a binding agent for binding a pathogen and a pair of transducers coupling the piezoelectric substrate to an operating system, wherein the detector is configured to detect a change in frequency of the piezoelectric substrate when the pathogen interacts with the binding agent. The pathogen can be present in the air or in a fluid. The system can be configured to continuously or periodically monitor air or fluid samples. Exemplary fluid samples include bodily fluids such as blood, saliva, urine, sweat, tears. Other fluids include aqueous or non-aqueous fluids, gases, potable water, waste water, and the like. In certain embodiments, the disclosed detection system can be placed inline with water distribution system, for example a municipal water distribution system.

In yet another embodiment, the detection system can be configured to continuously monitor for the presence of spores, cysts, protective spores, or reproductive spores. Representative spores include, but are not limited to bacterial and fungal spores. Exemplary bacterial spores, include but are not limited to endospores. In one particular embodiment the detection system is configured to detect Bacillus and or Clostridium bacteria or spores, and in particular Bacillus anthracis or spores from Bacillus anthracis. Exemplary fungal spores include, but are not limited to spores produced by Strachybotrys chartarum and more often as Strachybotrys atra or black mold. The detection system can be located in a living space, in a wall, or in a location suspected of containing either an airborne or waterborne pathogen.

Still another embodiment provides a system for detecting the presence of one or more predetermined target polynucleotides or nucleic acids. The detection system includes a detector, for example a detector comprising a piezoelectric substrate surface-modified with a binding agent for binding a target polynucleotide and a pair of transducers coupling the piezoelectric substrate to an operating system. In this embodiment, the binding agent is a nucleic acid complementary to the target polynucleotide. The binding agent can include polynucleotides having modified backbones to increase stability and resistance to degradation. In other embodiments, the detection system is configured for detecting at least two different polynucleotides in real-time.

The term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

In addition, polynucleotide refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

Term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

Some applications include the use of the disclosed systems in gene expression assays such as cDNA microarrays or oligonucleotides arrays. Hybridization would occur as has been described above; however, rather than detecting hybridization sometime after the event by imaging technologies, real-time detection of nucleic acid hybridization can be achieved. For example individual genes or gene fragments or oligonucleotides, tethered to the QCM platform could be monitored by the biosensor device. Further applications include the use of the disclosed systems for real-time detection of gene expression in research, or in medical examination to detect pathogens or marker molecules or in environmental monitoring to detect pathogens (viral or bacterial) or undesirable microbial species (e.g., bioterrorism, monitoring a living space or working space or theater of operation) or could be used in forensics to detect DNA in body fluids or monitor microbial populations post mortem.

### EXAMPLES

The gold surfaces of the QCM crystal were cleaned using Piranha solution (3 parts of 30% H₂O₂ in 7 parts H₂SO₄). The crystals were air-dried. (0.0234 g) of 3,3′-dithiopropionic acid was dissolved in 100% ethyl alcohol to make a 0.01M alcoholic solution. The solution was applied to the QCM gold electrodes and allowed to incubated overnight. The surface was washed 95% ethanol then aliquots of deionized water before allowing to air-dry. 1-Ethyl-3-(3-Dimethylamino-propyl) carbodiimide (EDC) (0.0133 mg) was dissolved in 0.1 ml of 1×TAE buffer. (0.0135 g) NHS was dissolved in 0.1 ml of buffer and mixed with EDC solution and the resulting mixture was incubated with the QCM surface for 30 min. The surface was washed with dI water and allowed to dry.

Mouse anti-Calmodulin IgG1 (20 ul of 100 ul/0.1 ml) (active against Plants and wide species variety) was incubated with the QCM gold electrodes for 6 hrs. The crystal was washed with 1×TAE buffer and allowed to dry. Ethanolamine (0.5M) was titrated with HCl to pH 8.0 before being applied to the quartz crystal. The surface was washed with dI water and allowed to air dry. FIG. 6 shows a line graph indicating the detection of calmodulin using the described device.

We claim:

1. A detection system comprising:
a chamber for holding culture media, the chamber comprising a cellular attachment surface; and
a detector disposed in the chamber comprising a piezoelectric substrate surface-modified with a binding agent for binding the target substance and a pair of electrodes coupling the piezoelectric substrate to an operating system,
wherein the detection system is configured to detect interaction of the target substance with the binding agent.

2. The system of claim 1, further comprising a second chamber for processing a sample for detection, wherein the second chamber is in fluid communication with the detector.

3. The system of claim 1, wherein the operating system processes acquired data and controls the detection system.

4. The system of claim 1, wherein detection occurs in a liquid or gaseous phase.

5. The system of claim 1, wherein the system is in fluid communication with a water distribution system or a heating, ventilating and air-conditioning system.

6. The system of claim 1, wherein detection of the target substance by the sensor system triggers a notification event.

7. The system of claim 6, wherein the notification event is based on a response algorithm that transmutes the binding event into a signal relayed to a central operating and control system.

8. The system of claim 7, wherein the signal is transmitted wirelessly.

9. The system of claim 8, wherein the system is in communication with a distributed computer network.

10. The system of claim 1, wherein the system is in gaseous communication with a water distribution system or a heating, ventilating and air-conditioning system, a wall cavity, or an enclosed living space.

11. The system of claim 1, wherein detection of the target substance triggers disinfection, decontamination, or removal of the target system.

12. The system of claim 11, wherein the a disinfecting amount of ultraviolet light is delivered to the chamber in which the target substance is detected.

13. The system of claim 1, wherein the detector is selected from an optical detection device, MEMS detection device, cantilevers and micromachined resonating structures, nanoparticle detection device, quantum dots or quantum piezoelectric dots, spectroscopic techniques or an acoustic wave detection device.

14. The system of claim 13, wherein the MEMS detection device comprises cantilevers, micromachined resonating structures or nanoparticle detection device.

15. The system of claim 14, wherein the nanoparticle detection device quantum dots or quantum piezoelectric dots.

16. The system of claim 1, wherein the target substance comprises a growth factor, differentiation inducing factor, polypeptide, vitamin, cofactor, nucleic acid, fatty acid, lipid, carbohydrate, or a combination thereof.

17. The system of claim 1, wherein the binding agent comprises a polypeptide, enzyme, nucleic acid, carbohydrate, lipid, a fragment thereof, or a combination thereof.

18. The system of claim 17, wherein the binding agent comprises an antibody or a fragment thereof.

19. The system of claim 1, wherein system is configured to detect the target substance in real-time.

20. The system of claim 1, wherein the system comprises more than one detector.

21. The system of claim 1, wherein the piezoelectric substrate is surface-modified with at least two binding agents specific for different target substances.

22. The system of claim 20, wherein at least one detector comprises a binding agent that is not specific for the target substance.

23. The system of claim 1, wherein the chamber comprises a cover having at least one port.

24. The system of claim 23, wherein the port provides fluid communication between the chamber and a reservoir.

25. The system of claim 24, wherein the reservoir comprises culture media, growth factors, differentiation inducers, pH buffer, antibiotics, or a combination thereof.

26. The system of claim 1, wherein the operating system comprises a computer, controller, processor, user interface, printer, monitor, power source, oscillator, software, or a combination thereof.

27. The system of claim 1, wherein the cellular attachment surface comprises a membrane, polymer, thermoplastic, plastic, collagen, metal, glass, mesh, fabric, scaffold, or a combination thereof.

28. A method for culturing cells, comprising:
incubating cells in a chamber containing culture media, the chamber comprising a cellular attachment surface;
detecting a target substance in the culture media with a detector disposed in the chamber, wherein the detector is surface-modified with a binding agent for binding a target substance; and
modifying culture conditions based on the presence or absence of the target substance detected in the culture media.

29. The method of claim 28, wherein the detector comprises a piezoelectric substrate surface-modified with a binding agent for binding the target substance and a pair of electrodes coupling the piezoelectric substrate to an operating system.

30. The method of claim 28, wherein the detector is selected from the group consisting of an optical detection device, MEMS detection device, nanoparticle detection device, and an acoustic wave detection device

31. The method of claim 28, wherein the cells are eukaryotic, prokaryotic or achaebacterial.

32. The method of claim 31, wherein the eukaryotic cells are plant cells or animal cells.

33. The method of claim 28, wherein the operating system automatically modifies culture conditions based on the presence of absence of the target substance in the culture media.

34. The method of claim 33, wherein the culture conditions are modified by adding a growth factor, differentiation inducing factor, cell adhesion factor, enzyme, lipid, carbohydrate, polypeptide, polynucleotide, antibiotic, pH buffer, acid, base, or a combination thereof.

35. The method of claim 33, wherein the culture conditions are modified by adjusting cell culture temperature.

36. The method of claim 28, wherein the cells are cultured under physiological conditions.

37. The method of claim 28, wherein the cells comprise embryonic cells.

38. A method for selecting cells comprising:
incubating cells in a chamber containing culture media, the chamber comprising a cellular attachment surface;
detecting a target substance in the culture media with a detector disposed in the chamber, wherein the detector is surface-modified with a binding agent for binding a target substance; and
selecting the cells in which the target substance is detected.

39. The method of claim 38, wherein the detector comprises a piezoelectric substrate surface-modified with a binding agent for binding the target substance and a pair of electrodes coupling the piezoelectric substrate to an operating system.

40. The method of claim 38, wherein the detector is selected from the group consisting of an optical detection device, MEMS detection device, nanoparticle detection device, and an acoustic wave detection device.

41. The method of claim 38, wherein the target substance is present in the culture media or on a cell surface.

42. The method of claim 38, wherein the target substance is a growth factor, polypeptide, polynucleotide, carbohydrate, differentiation inducing factor, neurotransmitter, lipid, cell surface protein, vitamin, intracellular component, or a combination thereof.

43. The method of claim 38, wherein the cells are eukaryotic, prokaryotic or achaebacterial cells.

44. The method of claim 43, wherein the eukaryotic cells are plant cells or animal cells.

45. The method of claim 38, wherein the cells comprise embryonic cells.

46. The method of claim 38, wherein the target substance is detected in real-time.

47. The method of claim 38, wherein the detector comprises at least two binding agents each specific for a different target substance.

48. A method for selecting cultures comprising:
monitoring culture media content of at least one cell culture with at least one detector disposed in each of the at least one cell cultures, wherein the at least one detector comprises a surface modified with a binding agent specific for at least one target substance; and
selecting the cell culture in which the at least one target substance is detected.

49. The method of claim 48, wherein the detector comprises a piezoelectric substrate surface-modified with at least one binding agent for binding at least one target substance and a pair of electrodes coupling the piezoelectric substrate to an operating system,

50. The method of claim 48, wherein the detector is selected from the group consisting of an optical detection device, MEMS detection device, nanoparticle detection device, and an acoustic wave detection device.

51. The method of claim 48, wherein the target substance is a biomolecule.

52. The method of claim 48, wherein the target substance is correlated with a growth stage of the cell culture, differentiation event of the cell culture, or ability of the cell culture to produce a specific tissue, specific cell type, or extracellular matrix.

53. The method of claim 48, wherein the target substance is secreted by at least one cell in the cell culture or displayed on a surface of at least one cell in the cell culture.

54. The method of claim 48, wherein at least one cell of the cell culture undergoes mitosis.

55. The method of claim 48, wherein the target substance interacts with the binding agent to modulate a resonance frequency of the piezoelectric substrate.

56. A system for detecting one or more target substances comprising:
(a) a piezoelectric substrate disposed in a culture chamber;
(b) a first and a second binding agent attached to a surface of the piezoelectric substrate, wherein the first binding agent specifically binds a first target substance and the second binding agent specifically binds a second target agent;
(c) an input transducer for converting an electric field into an acoustic wave and an output transducer for converting the acoustic wave to an electric field, wherein the input and output transducers are attached to the piezoelectric substrate; and
(d) an operating system in communication with the input and output transducers.

57. The system of claim 56, wherein the operating system detects binding of a target substance in real-time.

58. The system of claim 56, wherein the interaction of the first target substance with the first binding agent produces the second target substance.

59. A system for inline detection of a scaling agent comprising:
a detector comprising a surface modified with a binding agent for binding a scaling agent, wherein the detector is in fluid communication with a pulping system.

60. The system of claim 59, wherein the detector is selected from the group consisting of an optical detection device, MEMS detection device, nanoparticle detection device, and an acoustic wave detection device.

61. The system of claim 59, wherein the detector comprises a piezoelectric substrate surface-modified with a binding agent for binding a scaling agent and a pair of transducers coupling the piezoelectric substrate to an operating system.

62. The system of claim 59, wherein the scaling agent comprises hexenuronic acid, catechol, aluminum sulfate, derivatives thereof, or combinations thereof.

63. The system of claim 59, wherein the system is configured to detect the scaling agent in real-time.

64. A method for detecting a scaling agent in a pulping system comprising:
contacting a detector with a sample from the pulping system, wherein the detector comprises:
a piezoelectric substrate surface-modified with a binding agent for binding the scaling agent and
a pair of transducers coupling the piezoelectric substrate to an operating system,
wherein a change in frequency of the piezoelectric substrate is detected when the scaling agent interacts with the binding agent.

65. The method of claim 64, wherein the scaling agent comprises hexenuronic acid, catechol, aluminum sulfate, or combinations thereof.

66. The method of claim 65, wherein the binding agent comprises a antibody.

67. A system for detecting one or more target substances comprising:
(a) a detector;
(b) a first and a second binding agent attached to a surface of the detector, wherein the first binding agent specifically binds a first target substance and the second binding agent specifically binds a second target agent produced by the interaction of the first target substance with the first binding agent; and
(d) an operating system in communication with the detector.

68. The system of claim 67, wherein the detector is selected from the group consisting of an optical detection device, MEMS detection device, nanoparticle detection device, and an acoustic wave detection device.

69. The system of claim 67, wherein the second target substance comprises a growth factor, differentiation inducing factor, cell adhesion factor, enzyme, lipid, carbohydrate, polypeptide, polynucleotide, antibiotic, pH buffer, acid, base, or a combination thereof.

70. The system of claim 67, wherein the interaction of the first target substance with the first binding agent modifies the first target substance or the interaction of the second target substance with the second binding agent modifies the second target substance.

71. The system of claim 67, wherein the modification is selected from the group consisting of a conformational modification, a structural modification, and a covalent modification.

72. The system of claim 71, wherein the modification renders the first or second binding agent or the first or second target substance unable to interact with the detection system.

73. The system of claim 67, wherein the first or second target is degraded or covalently bound by the first or second binding agent.

74. A pathogen detection system comprising:
(a) a detector;
(b) a binding agent attached to a surface of the detector, wherein the binding agent specifically binds a pathogen or a fragment thereof; and
(c) an operating system in communication with the detector;
wherein the pathogen detection system is configured to detect the interaction of the pathogen with the binding agent in real-time.

75. The system of claim 74, wherein the detector is selected from the group consisting of an optical detection device, MEMS detection device, nanoparticle detection device, and an acoustic wave detection device.

76. The system of claim 74, wherein the pathogen comprises bacteria, fungi, protozoa, carcinogens, volatile organic compounds, viruses, prions, parasites, or a combination thereof.

77. The system of claim 74, wherein the pathogen comprises a spore.

78. The system of claim 74, wherein the spore is produced by black mold or Bacillus anthracis.

79. The system of claim 74, wherein the system is in fluid communication with a water distribution system or a heating, ventilating and air-conditioning system.

80. The system of claim 74, wherein the system is in gaseous communication with a water distribution system or a heating, ventilating and air-conditioning system, a wall cavity or an enclosed living space.

81. A piezoelectric array comprising:
a piezoelectric substrate with a plurality of regions surface-modified with a binding agent, wherein each region binds a specific target substance, and wherein the array is configured to detect interaction of more than one target substance with the binding agents.

82. The array of claim 81, wherein the piezoelectric substrate is coupled to an operating system.
